(19) United States
(12) Patent Application Publication
(10) Pub. No.: US 2010/0041989 A1
(43) Pub. Date: Feb. 18, 2010

(54) USE OF ULTRASOUND AS AN ANTIVASCULAR AGENT

(75) Inventors: Chandra Sehgal, Wayne, PA (US); Andrew K. W. Wood, Philadelphia, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: THE TRUSTEES OF THE UNIVERSITY OF PENNSYLVANIA, Philadelphia, PA (US)

(21) Appl. No.: 12/442,597

(22) PCT Filed: Oct. 1, 2007
(86) PCT No.: PCT/US07/80095

§ 371 Date: Mar. 24, 2009

Related U.S. Application Data
(63) Continuation of application No. 60/827,564, filed on Sep. 29, 2006.

(51) Int. Cl. A61N 7/00; A61B 8/00

(52) U.S. Cl. 600/439; 601/2

(57) ABSTRACT

A method for detecting and disrupting fragile blood vessels formed as a result of tumor angiogenesis by manipulating an extent of tissue vascularity in vivo, the method includes targeting a region in need of manipulating the extent of vascularity of the tissue by a diagnostic imaging; visualizing said fragile blood vessels in the tissue; obtaining a characteristic of the tissue; and contacting the tissue with a therapeutic ultrasound producing an unfocused or mildly focused ultrasound beam having an intensity below 2.6 W/cm2, wherein the therapeutic ultrasound is emitted from an ultrasound unit in accordance with the characteristic of the tissue and thereby reducing the extent of vascularity of the tissue and disrupting said fragile blood vessels formed to support the tumor in the region of interest.

### STATEMENT REGARDING FEDERALLY SPONSORED RESEARCH OR DEVELOPMENT

This research was supported in part by U.S. Government funds (Grant No. EB001713 from the National Institute of Health) and the U.S. Government may therefore have certain rights in the invention.

### SPECIFICATION

### Background of the Invention

1. Field of Invention

This invention relates to noninvasive or minimally invasive disruption of angiogenesis of tissues by ultrasound. More specifically, the invention relates to disrupting angiogenesis of tumor tissues by unfocused ultrasound having intensities of below 2.6 W/cm².

2. Description of Related Art

The use of ultrasound for clinical imaging as well as for its therapeutic actions in physical therapy is well known. Although the ultrasound energy in routine B-mode diagnostic ultrasound imaging causes minimal biological effects (Barnett et al. 2000), in physiotherapy, however, it is widely used to heat peri-articular structures and sites at bone-muscle interfaces. The physiotherapy ultrasound power levels generally range from 0.2 to 2.6 W/cm², the frequencies from 1 to 3 MHz, and in vitro temperature rises of 1 to 8° C. have been described (Baker et al. 2001; Demmink et al. 2003; Robertson and Baker 2001).

In combination with photodynamic therapy, a treatment that combines a light source and a photosensitizing agent, low intensity ultrasound (0.51 W/cm², 1 MHz, 10 min treatment time) was also used to inhibit the growth of cutaneous squamous cell carcinomas in mice, but the authors concluded that the mechanism of the response remained unclear (Jin et al. 2000).

In contrast to low intensity ultrasound, beams of high intensity focused ultrasound have been used to treat benign (Madersbacher et al. 1997) and malignant (Uchida et al. 2002) prostate cancer as well as hepatic (Rowland et al. 1997) and oesphageal tumors (Melodelima et al. 2003).

U.S. Patent Application Publication 2005/0165298 to Larson et al. described utilizing high intensity focused ultrasound to induce angiogenesis.

Angiogenesis is the process by which new vessels grow toward and into a tissue. Angiogenesis is required for several physiologic processes including embryogenesis, corpus luteum formation, and wound healing. It is also a critical element in the pathogenesis of many disorders, most notably rapid growth and metastasis of solid tumors.

The formation of new blood supplies is essential to the unrestricted growth of tumors. Tumors do not produce their own new blood vessels, but for nutrients and oxygen rely on vascular supplies derived from the nearby host tissue. It has been shown that tumors can attain a size of only 1-2 mm by simple diffusion of nutrients, but can exist for an extended period in this quiescent, static, prevascular stage before angiogenesis is “switched on (Folkman, J., J. Nat'l Cancer Inst. 82:4 (1989)). Once angiogenesis is upregulated, the tumor enters the vascular phase allowing for exponential growth and resultant clinical manifestations. Although the mechanism of “switching on” is not known, once the switch has been thrown, angiogenic factors elaborated by the tumor and tumor-associated inflammatory cells interact with endothelial cells in neighboring capillaries to stimulate new capillary beds and to prepare the local environment for their in-growth.

It has been assumed that angiogenesis correlates with tumor aggressiveness. This assumption has been supported in clinical trials investigating a variety of tumor types.

Inhibiting angiogenesis as a method of inhibiting cancer tissue has been investigated. As a tumor grows, the upregulation of angiogenic factors results in the sprouting of new blood vessels from pre-existing vessels to supply the tumor, but these new vessels fail to mature into a normally functioning vasculature (Carmeliet and Conway 2001; Carmeliet and Jain 2000; Folkman 1971; Folkman 2001; Haroon et al. 1999; Jain 2002). The vessels are fragile and leaky; their endothelial cells remain loosely associated, there is continued degradation of the extracellular matrix, and the basement membrane is discontinuous or may fail to form. The resulting vasculature is not fully functional has a non-uniform distribution and demonstrates irregular branching and arterio-venous shunts (Carmeliet and Conway 2001; Haroon et al. 1999; Jain 2002).

Concurrent magnetic resonance imaging has also been used to monitor the rise in temperature during insonation of implanted carcinomas in rabbits (Palussiere et al. 2003) and fibroadenomas of the human breast (Hynynen et al. 2001). High intensity beams cause the tissue temperature to rise above 60° C. and to as much as 90 to 95° C., and cause permanent damage to tissues related to a localized instantaneous coagulative necrosis (Clement 2004; Diederich et al. 2004; Uchida et al. 2002). The ultrasound intensity generally ranges from 103 to 10⁴ W/cm², with frequencies from 0.5 to 10.0 MHz, although 1.5 MHz is most commonly used for a treatment time of 1 to 30 s (Clement 2004).

Some researchers have utilized lower intensities (2.6 to 26.7 W/cm²) for the treatment of experimental canine prostatic neoplasia (Hazle et al. 2002) and human oesophageal cancer (Melodelima et al. 2003).

Low intensity ultrasound (˜1 to 5 W/Cm²) has been evaluated for cancer therapy (Rosenthall et al 2004; Tomizawa et al 2001; Yu et al 2004). In these studies, the tumor growth was suppressed by combining anticancer agents with ultrasound. While ultrasound treatments have targeted tumor cells, the role of low level ultrasound to disrupt tumor vasculature was not recognized.

Ultrasound had been used to deliver therapeutic agents to tumors (see, for example, U.S. Pat. No. 6,812,204 to McHale et al.

Ultrasound had been used to hyperthermically treat tumor cells in soft tissues (see, for example, U.S. Pat. No. 4,646,756 to Watmough et al.).

Combining using high intensity focused ultrasound (HIFU) waves with ultrasound imaging has been described in U.S. Pat. Nos. 5,471,988; 5,769,790; 5,895,356; and 6,716,184.

U.S. Pat. No. 5,471,988 to Fujio et al. describes combining HIFU and imaging on one probe.

U.S. Pat. No. 5,769,790 to Watkins et al. describes combining B-mode ultrasound imaging (i.e., low-intensity imaging) for visualization of focal spot of HIFU with tissue B-mode image to guide HIFU energy to a desired focal spot in the tissue.

U.S. Pat. No. 5,895,356 to Andrus et al. describes a two-step approach of combining imaging and HIFU for targeting high energy at the desired location. In the first step, imaging is used to locate HIFU focal point and the information is stored in the memory of a computer. The location information is subsequently used to target high energy to the target area.

U.S. Pat. No. 6,716,184 to Vaezy et al. describes a method and apparatus for the simultaneous and in real time use of ultrasound on a probe for imaging and therapeutic purposes using high intensity focused ultrasound (HIFU) waves to cause lesions in blood vessels so that the supply of nutrients and oxygen to a region such as tumor is interrupted. HIFU therapy employs ultrasound transducers that are capable of delivering 1,000-10,000 W/cm² at a focal spot in contrast to diagnostic ultrasound where intensity levels are usually bellow 0.1 W/cm². U.S. Pat. No. 6,716,184 describes an apparatus which combines both imaging and therapeutic ultrasonic transducers for simultaneous imaging and treatment of a tumor. An external trigger electronic pulse is used to synchronize the imaging and therapeutic probes. The problem with simultaneous imaging is that the therapy pulse introduces artifact in the image. It is proposed that these artifacts can be controlled by shifting the time delay between imaging pulse or image frame and the therapy pulse such that the artifacts do not overlap the target region. Notably, in this approach, the therapy pulse has to be small to allow real time visualization. If the therapy pulse is long (which is often desirable to deliver large amount of energy to the focal site), then imaging can not be performed for extended time period, thereby losing the real time application. In the case of continuous therapeutic wave, the method will not work.

Even though HIFU waves of U.S. Pat. No. 6,716,184 are used at frequencies within the range of 0.5 MHz to 10 MHz which is comparable to frequencies used in the present invention, the intensity of the therapeutic ultrasound is drastically lower. Specifically, in the present invention the intensity of the therapeutic ultrasound is below 2.6 W/cm². Significantly, U.S. Pat. No. 6,716,184 did not recognize that blood vessels can be disrupted at much lower intensities, which would not ablate or cause damage to surrounding tissue.

Further, the present invention utilizes a continuous ultrasonic wave and not pulsed wave as in U.S. Pat. No. 6,716,184, which in turn imposes significantly less damage to the surrounding tissue and disrupts tumorous tissue blood vessels only.

In summary, the above described patents have one common specific aim for imaging, which is to identify a location in the tissue where HIFU focal spot must be placed to target high intensity acoustic energy. The imaging is also used to monitor the extent of damage, although there is no clear definition of how the damage to the tissue is measured or evaluated. Often, a transient change in tissue echogenicity is mentioned but it is not made clear how this information is used to evaluate the effectiveness of the therapy.

None of the references teach the use of low-intensity ultrasound for therapy. None of the references teach how ultrasound imaging can be used to measure tissue property (e.g., tumor vascularity or the extent of tumor perfusion) and how to use the image-derived information to determine the dose of ultrasound energy that must be delivered to the desired site.

Despite the foregoing developments, it is desired to provide methods and apparatuses for noninvasive or minimally invasive disruption of angiogenesis of tissues using ultrasound having intensities of below 2.6 W/cm².

All references cited herein are incorporated herein by reference in their entireties.

### BRIEF SUMMARY OF THE INVENTION

Accordingly, one aspect of the invention comprises a method for detecting and disrupting fragile blood vessels formed as a result of tumor angiogenesis by manipulating an extent of tissue vascularity in vivo, the method comprising: targeting a region in need of manipulating the extent of vascularity of the tissue by a diagnostic imaging; visualizing said fragile blood vessels in the tissue; obtaining a characteristic of the tissue; and contacting the tissue with a therapeutic ultrasound producing an unfocused or mildly focused ultrasound beam having an intensity below 2.6 W/cm², wherein the therapeutic ultrasound is emitted from an ultrasound unit in accordance with the characteristic of the tissue and thereby reducing the extent of vascularity of the tissue and disrupting said fragile blood vessels formed to support the tumor in the region of interest. Ultrasound could be used alone or in conjunction with chemical agents that enhance tissue heating and cavitation activity.

Another aspect of the invention comprises a method of treating a tissue with ultrasound combined with administering antivascular agents (e.g., anti-angiogenic drugs such as, for example, AVASTIN (Genen tech, San Francisco, Calif.) and optionally using a sensitizer agent as described, for example, in U.S. Pat. No. 6,498,945 to Alfheim et al.).

Another aspect of the invention comprises a device for detecting and manipulating tissue vascularity, the device comprising:

an imaging unit for imaging of an area of a tumor;

a processing unit for determining a characteristic of the tumor;

a processing unit for determining a characteristic of an ultrasound treatment correlated to the characteristic of the tumor; and

an ultrasound treatment unit capable of generating an unfocussed or mildly focused ultrasound beam having an intensity below 2.6 W/cm² which is sufficient to disrupt blood vessels formed to support the tumor but not blood vessels formed to support healthy tissues.

### BRIEF DESCRIPTION OF SEVERAL VIEWS OF THE DRAWINGS

The invention will be described in conjunction with the following drawings in which like reference numerals designate like elements and wherein:

FIG. 1 is a flow chart demonstrating steps of the method of the invention.

FIG. 2 is a block diagram of the ultrasound apparatus for disrupting tissue vascularity.

### DETAILED DESCRIPTION OF PREFERRED EMBODIMENTS OF THE INVENTION

The invention is based on the discovery that the extent of tissue vascularity can be manipulated with a low intensity ultrasound (LIU). Particularly, it was observed that while the healthy vessels were not disturbed, the leaky, fragile vascular channels formed as a result of tumor angiogenesis were disrupted after a treatment with an unfocused or mildly focused ultrasound beam at intensities below 2.6 W/cm². Disrupting the tumorous tissue vascularity is effective in treating tumors. The invention is applicable to various cancers such as, for example, skin cancer and other cancers affecting internal and external areas of a body, e.g., kidneys, prostate, uterus, breast cancer, etc.

The invention relates to a method for detecting and disrupting tumorous tissue vascularity with high sensitivity and specificity using ultrasound vibrations at intensities below 2.6 W/cm².

In one aspect, the invention is a method of treating a tissue with ultrasound combined with administering antivascular agents (e.g., anti-angiogenic drugs such as, for example, AVASTIN (Genentech, San Francisco, Calif.) and optionally using a sensitizer agent as described, for example, in U.S. Pat. No. 6,498,945 to Alfheim et al.). In certain embodiments, chemotherapeutic compounds are not used.

### DEFINITIONS

The term “imaging” refers to a method of examining tissue by exposing the tissue to energy waves and measuring the differences in absorption of the energy transmitted or by measuring the energy scattered by the tissues or by measuring the release of energy by the tissues in the presence of the energetic waves. Contemplated imaging systems include but are not limited to: magnetic resonance imaging (MRI), computerized tomography scanning, positron emission tomography scanning, radionuclide scanning; conventional X-ray and ultrasound imaging (e.g., using contrast agents). Exemplary imaging systems are described in U.S. Pat. Nos. 6,280,383 to Damadian, 6,009,342 to Brasch et al, 6,694,171 to Breskin et al., U.S. patent application Publication Nos. 20040181152 to Zhang et al, 20060216238 to Manchester et al. and 20060020205 to Kamiyama). Methods based on using ultrasound for detecting and characterizing a tumor and measuring vascularity are described in U.S. Pat. Nos. 6,858,011 to Sehgal, 6,728,567 to Rather et al, 6,716,184 to Vaezy et al., 5,471,988, 5,769,790 and 5,895,356 as well as in Example below.

The term “low intensity ultrasound (LIU)” as used herein refers to an unfocused or mildly focused ultrasound beam at intensities below 2.6 W/cm² which is strong enough to disrupt the leaky, fragile vascular channels formed as a result of tumor angiogenesis while the healthy vessels were not damaged. In certain embodiments, LIU intensities are in the range of 0.01 W/cm² to 2.6 W/cm². In other embodiments, LIU intensities are below 1.0 W/cm². The duration of the treatment can be prolonged to achieve the desired degree of disruption. LIU of the present invention is applied as continuous waves (e.g., duration is measured in minutes or hours) as well as long tone bursts.

The term “unfocused or mildly focused ultrasound beam” refers to a beam with degree of focusing (κ) of value 6 or less. For an ultrasound wave of wavelength (λ) generated by a transducer of radius (r) and focal length (F), degree of focusing (κ) is defined as the ratio of near-field depth to focal length as follows:

$$κ = \frac{{r}^{2} / λ}{F} .$$

The term “ultrasound beam characteristics” as used herein includes beam size, ultrasound frequency, continuous or pulsed dose, ultrasound intensity, treatment time (on/off duration of treatment).

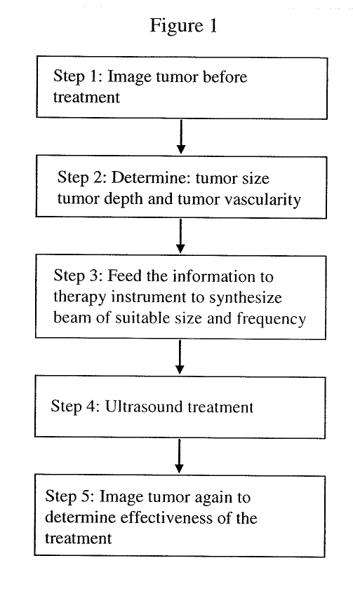

FIG. 1 is a flow chart depicting the steps of the method for detecting and disrupting tumor blood vessels in vivo.

In Step 1, the tumor under study is visualized using a suitable imaging system (an imaging unit) that is capable of visualizing tissue vascularity. The imaging will be optimized to visualize small vessels of the tumor. This would involve acquiring the images at low-ultrasound exposure. The low-ultrasound exposure will be achieved by either acquiring the images at a slow frame rate or by altering the image acquisition properties (viz. line density, and pulse echo characteristics of the imaging pulse).

In Step 2, the information obtained from Step 1 is used to determine the size of the tumor, depth of the location of the tumor from the skin and the extent of vascularity of the tumor in a processing unit for determining a characteristic of the tumor. A microprocessor or a similar device can be used for processing this information. The selection of such devices and relevant algorithms will be apparent to a person skilled in the art of imaging tissues (see, for example U.S. Pat. No. 6,872,180 to Reinhardt et al.). In a preferred embodiment, an algorithm described in U.S. Pat. Nos. 6,858,011 to Sehgal is used. In one embodiment, a user will define a fraction of the tumor area and a microprocessor will measure the area enhanced by a contrast agent. The ratio of the two will be a measure of a fraction of tumor with active vasculature and a larger area would require a larger duration and/or intensity of the ultrasound.

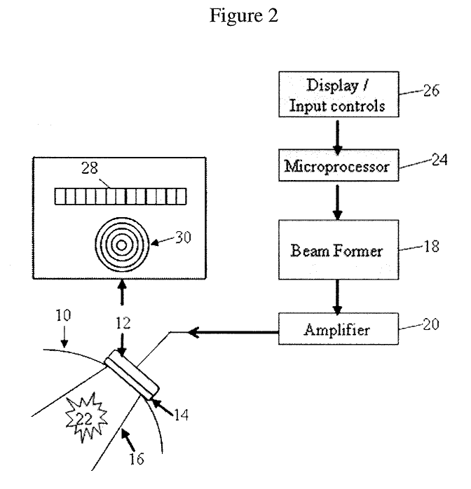

In Step 3, the information obtained in Step 2 is delivered to a processing unit for determining a characteristic of an ultrasound treatment correlated to the characteristic of the tumor (e.g., a microprocessor or a similar device) to select the size of the ultrasound beam, frequency of sonication, on/off cycle and the treatment time for an ultrasound treatment device that must be used to disrupt tumor vasculature (shown in FIG. 2). Selection of such devices and relevant algorithms will be apparent to a person skilled in the art of imaging tissues.

In Step 4, based on the instructions from Step 3, the ultrasound treatment unit generates an unfocussed or mildly focused ultrasound beam having an intensity below 2.6 W/cm² which is directed towards and applied to the area of the tumor. The ultrasound beam covers the area of the tumor and is aimed at the leaky, fragile vascular channels whose formation arose as a result of tumor angiogenesis. The duration of the treatment depends on the degree of disruption required. In certain embodiments, the degree of disruption is at least 10%, 20%, 30%, 40%, 50%, 60%, 70%, 80%, 90%, or 99%.

In Step 5, after completing the treatment, the tumor vasculature is visualized again to determine the change in tissue vascularity and decide whether a follow up treatment is needed.

In another aspect, the invention is a device for detecting and manipulating tissue vascularity, the device comprising: an imaging unit for imaging of an area of a tumor; a processing unit for determining a characteristic of the tumor; a processing unit for determining a characteristic of an ultrasound treatment correlated to the characteristic of the tumor; and an ultrasound treatment unit capable of generating an unfocussed or mildly focused ultrasound beam having an intensity below 2.6 W/cm² which is sufficient to disrupt blood vessels formed to support the tumor but not blood vessels formed to support healthy tissues.

In the apparatus of the invention, the imaging step (Step 1), the image-derived information processing steps (Steps 2, 3 and 5) and the treatment step (Step 4) can be conducted by using separate devices or integrated into one device.

FIG. 2 depicts a block diagram of an ultrasound treatment device for disrupting tissue vascularity that is used with tumor visualization performed by a separate imaging instrument. In other embodiments, a single unit serving as an imaging unit and a treatment unit is utilized (not shown).

An ultrasound transducer (12) is coupled to the treatment area through coupling gel and an intervening cooling jacket (14). An unfocused ultrasound beam (16) is generated by beamformer (18) and an amplifier (20) to insonate tumor volume (22). The ultrasound parameters, intensity, duty cycle, sonication duration and time of intermittent imaging, are controlled by a microprocessor (24) connected to a display and input control device (26). The transducer can be a single element if the size of the tumor and its location are known. In a preferred embodiment the transducer is an array of linear or circular elements whose functionality and timing of excitation are controlled by the microprocessor (24) and the beamformer (18). The cross-section view of two array transducers is shown by the diagrams enclosed in the dotted box.

From ultrasound images, the microprocessor (24) will calculate the area of perfusion and map the area of the tumor that needs to be sonicated. The ultrasound beam must be targeted to the tumor, which involves optimizing the beam with respect to the following:

(a) size of the beam to control the region to be sonicated; this will also involve calculating temperature distribution produced by the beam over the region of tumor vascularity. Bioheat transfer equation in combination with the images will be used to perform the calculations;

(b) frequency (determined primarily by the depth of the tumor). Deeper located tumors would require lower sonication frequency for increased penetration;

(c) intensity and duration of sonication. Larger and highly vascular tumors will require higher intensity and longer treatment time;

(d) on/off duty cycle for treatment. Use of high intensity can potentially produce unwanted tissue damage. In such cases it will be desirable to use longer “off” times in between the “on” treatment times.

It is contemplated to conduct imaging and therapeutic treatment sequentially as well as simultaneously with imaging using apparatuses as described in U.S. Pat. No. 6,716,184 to Vaezy et al., but modified to use LIU instead of HIFU with frequencies of imaging and therapeutic ultrasound adjusted not to overlap. In the present invention, due to the continuous or long tone burst ultrasound LSU wave, the approach of shifting the time delay between imaging pulse (or, image frame) and the therapy pulse to reduce artifacts in the image will not function. In the sequential mode, imaging will be followed by therapy, whereas in the simultaneous mode therapy and imaging will be performed together with a constraint that the ultrasound frequency content of the imaging pulse does not overlap the therapeutic frequency. For example, therapeutic ultrasound can be performed at 1-3 MHz, while the imaging ultrasound can be performed at 5-7 MHz.

Based on the teaching of the invention, a person of ordinary skill in the art will be able to select necessary building blocks for manufacturing the device of the invention from variety of mechanical, electrical, and software parts using, for example, patents described herein.

The frequency of sonication ranges from 20 KHz to 20 MHz. In the on/off mode, the duty cycle ranges from 0.1 to 1. The ultrasound intensities (spatial average temporal average, ISATA) are selected such that the healthy vessels are not disrupted by the treatment and range from 0.01 W/cm² to 2.6 W/cm², preferably the ultrasound intensity is below 2.6 W/cm². The total exposure time vary from few seconds to an hour depending on the ultrasound intensities used. The choice of various parameters is guided by imaging steps described in FIG. 1.

The sonication procedure could be a single treatment or multiple treatments with intermittent cessation of sonication. The on/off time of the therapy system is controlled by suitable electronic devices and the microprocessor encased in a portable enclosure. To avoid excessive heating of the transducer element at the skin-transducer interface, the transducer is enclosed in a jacket containing cooling fluid. Under the condition of low ultrasound intensities and other exposure related safeguards controlled by the microprocessor, the ultrasound treatment device could potentially be used for treatment in a small private clinic or a hospital.

Devices of the present invention include surface treatment apparatuses and internal use apparatuses and can be made in variety of shapes to deliver treatment to a desired area. Non-limiting examples of such apparatuses include a wand, a paddle, a catheter, a vaginal probe, and a rectal probe. Further, the device of the invention can be attached to a body with help of fasteners (e.g., a sleeve) and left in place for the total treatment time or it can be held in place manually.

The invention will be illustrated in more detail with reference to the following Examples, but it should be understood that the present invention is not deemed to be limited thereto.

### Example

Utilizing power Doppler ultrasound imaging together with an ultrasound contrast agent, the effect of physiotherapy ultrasound on tumor perfusion was investigated. In laboratory animals, intravenously injected ultrasound contrast agents have previously been used to visualize blood flow in tumor microvessels (Chomas et al. 2003; Fleischer 2000; Forsberg et al. 2004; Kamotani et al. 2003; Krix et al. 2003). Also, differences in perfusion between regions in a tumor have been demonstrated (Chomas et al. 2003) and quantified using image-gating methods (Kamotani et al. 2003).

Murine tumor model General anesthesia was induced in 22 female mice (6-8 weeks of age; 20-25 g body weight; C3HV/HeN strain; Charles River Laboratories, Wilmington, Mass., USA) by the intraperitoneal injection of ketamine hydrochloride (54 mg kg⁻¹; Abbott Laboratories, N. Chicago, Ill., USA) and xylazine hydrochloride (4 mg kg⁻¹; Phoenix Pharmaceutical Inc, St Joseph, Mo., USA). K1735²² murine melanoma cells (syngeneic with C3H/HeN mice) were cultured at 37° C. in 5% CO₂ maintained in Dulbecco's Modified Eagle's Medium (Cambrex Corporation, Walkersville, Md., USA) supplemented with 10% fetal bovine serum (Hyclone Inc, Logan, Utah, USA) and 1% penicillin/streptomycin (Invitrogen Corporation, Grand Island, N.Y., USA). One or two million melanoma cells were injected subcutaneously in the right flank of each anaesthetized mouse. All the animals were imaged before and after tumor insonation and histology was performed in a selected number.

Prior to the ultrasonographic studies, a tail vein was catheterized (26 gauge Abbocath, Abbot Ireland, Sligo, Republic of Ireland). Each mouse was transferred to an acrylic box and general anesthesia was induced with 2% isoflurane (Isosol, Halocarbon Laboratories, River Edge, N.J., USA) and air. The animal was then placed on a table under a heat lamp; a facemask was applied and anesthesia was maintained with 1.5% isoflurane during the periods of ultrasonographic observation.

Within 30% of the completion of the experiment, a thoracotomy was performed in 15 anaesthetized mice, the right atrium was incised and, in order to fix the tissues, 10 mL 4% w/v parafoimaldehyde (Fisher Scientific, Fair Lawn, N.J., USA) in phosphate buffered saline and sodium hydroxide (20 μL) were injected into the left ventricle. The tumor and overlying cutaneous tissues as well as the adjacent thigh muscles were removed for histopathologic study. In the remaining seven anaesthetized mice only the ultrasound studies were performed (as described in the next sections).

Ultrasonography and ultrasonographic contrast medium In each anaesthetized mouse, ultrasonographic observations were made of the tumor before and after its insonation. A depilatory cream (Surgi-Prep, Sparta Surgical Corporation, Concord, Calif., USA) was used to remove the hair coat from the tumor site and ultrasound coupling gel was applied to the skin. B-mode ultrasonographic observations using an identical time gain compensation for all animals and including compound imaging, (see Shattuck and von Ramm 1982) of the tumor were made (7-15 MHz probe; foot print=10×35 mm; HDI 5000 SonoCT, Philips, Bothell, Wash., USA); the dimensions of the tumor were measured. Images were made in the dorsal and sagittal anatomical planes and recorded on VHS-videotape.

At the completion of each B-mode study, an ultrasonographic contrast medium was injected into the tail vein (0.1 mL perflutren protein-type A microspheres; Optison, GE Healthcare, Princeton, N.J., USA), and the enhancement of power Doppler images, using a mechanical index of 0.9, was recorded on a videotape for quantitative analysis. The microbubbles contained in the contrast agent were destroyed by the ultrasound imaging pulses. To maximize the enhancement of the power Doppler images, the exposure of the contrast medium to ultrasound was reduced by acquiring the images at a low frame rate of 0.5 Hz (achieved by gating the ultrasound scanner at 0.5 Hz). The Doppler ultrasound imaging was continued until there was no enhancement in the tumor—thus ensuring that no microbubble-related effects occurred during the insonation of the tumor.

Tumor insonation. Once the tumor had grown (over three to four weeks) to a minimum size of 1 cm in at least one dimension, the tumor was insonated with a physiotherapy ultrasound machine (1 MHz, continuous output, power level=2; D150 Plus, Dynatronics Corp., Salt Lake City, Utah, USA). The diameter of the transducer was 2.5 cm and the manufacturer stated that, utilizing the selected parameters, the intensity (spatial-average-temporal average, [I_{SATA}]) of the ultrasound beam was 2.0 W/cm². The effective intensity of the ultrasound beam was measured using a radiation force balance (UPM 30, Ultrasound Power Meter, Ohmic Instruments Co, St Michaels, Md., USA). The degassed water used in the balance was prepared by boiling IL deionized water in a beaker for 30 min. The beaker was sealed and cooled to room temperature. The measured ultrasound intensity (I_{SATA}) was 2.28±0.02 W/cm².

To prevent the possible interference from the microbubbles in the contrast agent, there was at least a five min period between the completion of the contrast enhanced Doppler imaging and the insonation of the tumor. To ensure that the animal did not move during insonation, the isoflurane was increased to 2%. To avoid insonating the normal contiguous tissues, a rectangular-shaped foam pad (5.0×3.0×0.7 cm) with a 1 cm diameter hole was applied to the skin of each anaesthetized mouse with the hole centered over the tumor. The hole was filled with non-degassed ultrasonographic coupling gel; part of the insonating beam was transmitted through the gel to the tumor, but other parts of the beam were prevented from reaching the normal tissues contiguous with the tumor by the air-filled foam. The mouse was placed in ventral recumbency and a sagittal anatomical plane was used for the insonation. In this anatomical plane the insonating beam was directed vertically and passed through the tumor in a dorso-ventral direction towards the table top, and not through the abdominal wall and the adjacent organs in the peritoneal cavity. The B-mode and power Doppler observations were made in the identical anatomical plane. Any effects of insontion on the mouse, either locally on the tumor or generally on the health of the animal, were recorded.

In groups of six animals, the tumor was insonated (2.28 W/cm², 1 MHz, continuous wave) for either one, two or three min. When the insonation time exceeded one min, there was a gap of five min between each one min period of insonation, during which the face of the probe was cooled in tap water at room temperature. A further group of 4 animals acted as controls—the sonographic probe was applied to the tumor for a total of three min with five min intervals between each one min of application as described above, but the machine was not switched on. In each mouse, the ultrasonographic observations (B-mode and contrast studies) were made prior to and within five min of insonation or sham insonation, and were repeated 24 h later.

Analysis of data. The ultrasonographic images of each tumor, recorded on videotape, were digitized (30 frames s⁻¹; 24 bit; Adobe Premiere 6.5, Adobe Systems Inc, San Jose, Calif., USA) using a digitizer (MediaConvertor, DVMC-DA2, Sony, Tokyo, Japan) connected to a standard personal computer and stored in an uncompressed format. Before and after the insonation, B-mode and power Doppler images of each tumor were selected for analysis. The power Doppler images were made prior to and following the injection of the ultrasound contrast agent; the latter images were chosen on the basis of the one showing the maximum color enhancement. To accurately locate the tumor in the post-contrast images, a region of interest was traced around the boundaries of the tumor as shown in the pre-contrast image and it was then superimposed on the post-contrast image. The images were analyzed using a custom-made software program written in an interactive data language (IDL, Research Systems Inc., Boulder, Colo., USA). The program has been described (Sehgal et al. 2000, 2001). In summary., it involved using the color palate in the power Doppler image to form a look-up table for the color scheme in hue-saturation-value (HSV) space. The lowest to highest values in the color palate were assigned values between 0 and 100 on a linear scale. Then the computer searched for colored pixels within the region of interest and assigned each pixel a value between 0 to 100, depending on the value it corresponded to in the calibrated color palate of the ultrasound image. This analysis provided a histogram of the region of interest, where the zero value represents grayscale pixels with no Doppler signal, and values 1-100 represented the strength of Doppler signal.

Cumulative histogram curves were obtained for both the treated and control groups of mice by plotting the color level against the fraction of colored pixels. Each curve fractionates the area of the rectangle between (0, 0) and (100, 1) into vascular and avascular components (Appendix A). Therefore, the difference between the area under the curve after treatment and that before treatment was used as a measure of the antivascular effect of insonation in the tumor. The null hypothesis, that there was no reduction in the vascular area between pre- and post-treatment groups, was tested with a paired t-test (Medcalc Software, Mariankerke, Belgium); the difference was considered significant when p<0.05. An unpaired t-test was used to compare the post-treatment area of the control animals with the post-treatment areas of each of the three treatment groups; the difference was considered significant when p<0.05.

The average difference (mean±standard error) between the areas under the pre- and post-insonation histogram curves for the controls and each treatment time was used to evaluate a dose/response relationship by a linear regression analysis. Tumor vascularity was also compared (t test) between observations made immediately after insonation and one day later.

Estimation of ultrasound heating. An estimation of the heating of mammalian tissues was provided by observations made in 1 cm thick slices of fresh bovine muscle. Each of three muscle samples, initially at room temperature, was subjected to 3×1 min periods of insonation as described above. A digital thermometer with thermistor sensor (Thermometer 5800, Omega Engineering Inc, Stamford, Conn., USA; diameter=1.5 mm; response time <800 ms) was inserted into the muscle prior to and after (but not during) each period of insonation and the temperature was recorded. We did not use thermocouples (implanted into the tumor) to make in vivo measurements of the temperature increase, as we wished to avoid the complication of the conversion of acoustic energy to heat that would occur at a tumor-thermocouple interface.

Histological studies. In 15 mice (three controls, and four for each of the three treatment times) the tumors were fixed overnight in 4% paraformaldehyde and were then embedded in paraffin, sectioned, and stained with hematoxylin and eosin. Each histological specimen was viewed under a microscope (Nikon E600 Eclipse, Nikon, Melville, N.Y., USA) for qualitative assessment of the tissue changes.

The mice recovered normally from the general anaesthesia used during the insonation of the tumor, were bright and alert, and ate and drank normally. Each tumor was clearly detectable in the B-mode images. It was generally hypoechoic to the surrounding structures and its boundaries were distinct. Immediately following insonation, the skin overlying the tumor blanched and in the mice receiving three min of insonation it was firm to touch. In some animals (n=10) a localized area of skin ulceration developed, but there was no apparent correlation between the treatment time and the presence of ulceration. Histologically, the skin overlying the tumor was inflamed and there was merging of collagen fiber bundles. In six mice, where the tumor was in close proximity to the femur, there was partial loss of function of the adjacent pelvic limb. At the bone/soft tissue interface between the femur and proximal muculature of the pelvic limb, histology demonstrated localized necrosis of muscle with an associated decrease in muscle striations, interstitial hemorrhage and vascular congestion. The mean peak rise in temperature after each 1 min sonation was 7.6±2.7° C. (n=9), corresponding to an average temperature increase of 3.8° C. over the duration of each one min period of insonation.

Prior to insonation, the contrast enhanced power Doppler images clearly demonstrated the normal vascular perfusion of the tumor—it was highly vascular as shown by the uniform flush of colour that filled the entire lesion; the surrounding normal tissues were also highly perfused. The post-insonation Doppler images showed, however, that the contrast medium did not enter all regions of the tumor, indicating that parts of the tumor were now avascular and had lost their normal vascular perfusion. For each treatment time these Doppler observations were quantified by plotting cumulative histogram curves which showed the percentage fraction of image pixels that were colored at each color level. The curves demonstrated that with increasing insonation there were fewer colored pixels in the image, whereas in the control animals the number of colored pixels was maintained.

Prior to insonation, all but 7% of the tumor was perfused (mean of pre-insonation data from all tumors). Following insonation of all mice, there were increases in the size of the avascular area in the tumor. When the post-treatment control animals were compared with each of the treatment groups the difference were highly significant (control versus one min−p=0.043; control versus two min−p=0.002; control versus three min−p<0.001). The avascular area in tumors receiving three min treatment had increased to 82%. A linear regression analysis showed that each min of insonation lead to a 25% reduction in tumor vascularity, confirming the potent antivascular effect of insonation. Post-insonation, normal vascular perfusion was maintained in the adjacent tissues, including the right kidney; the blood vessels adjacent to the tumor had retained their normal structure and function. One day following insonation, the avascular area in each treated group increased compared to the avascular area immediately after insonation, but the increase was small and was not statistically significant (one min−p=0.180, two min−p=0.429, three min−p=0.264). There also no significant change in the vascularity of the control group (p=0.176). The normal perfusion of tissues adjacent to the tumor was again observed.

The histopathological studies showed that the effects of insonation were predominantly on the vascular structures within the tumor and correlated with the findings in the contrast enhanced power Doppler ultrasonographic images. In each of the insonated mice, there was disruption of the walls of the tumor blood vessels with associated hemorrhage, vascular congestion and subsequent thrombosis; oedema was also present. The hemorrhage was more prominent in tumors receiving two or three min of insonation. In each mouse, there were fewer tumor cells and cell necrosis had occurred in the areas of vascular congestion and thrombosis.

A commercially available physiotherapy ultrasound machine was used to insonate the murine tumor. Such machines produce an ultrasound beam with a low intensity, are used as a routine in clinical practice, and no adverse bioffects on human tissues have been reported. Power Doppler and histopathologic observations showed that tumor blood vessels were disrupted by the beam of low intensity ultrasound. Doppler observations on the day following insonation demonstrated a continuing reduction in tumor vascularity, suggesting that the damage to the tumor blood vessels was long lasting. Also the post-insonation tumor cell necrosis was probably secondary to ischemia as it occurred in the areas of vascular congestion and thrombosis. The fragile, poorly functioning tumor vessels, whose formation is activated by the tumor's “angiogenic switch”, are likely to be more sensitive to insonation. Similar vascular congestion, thrombosis, and rupture to that found in the above study were reported in murine thigh tumors heated to 44° C. in a water bath for 30 min (Nishimura et al. 1988), and also in rat gliomas treated with combretastatin A-4 (Eikesdal et al. 2001). Recent studies have shown that the maturity of blood vessels within a tumor varies and that the more immature vessels are more sensitive to antivascular therapies (Gee et al. 2003). Since the ultrasound induced antivascular effects are unlikely to be dependent upon specific biochemical pathways used by various drug therapies, it is believed that ultrasound could be used to disrupt vessels of differing maturities.

Since the morphological changes observed in this study are similar to those caused by hyperthermia and combretastatin (not involving heat) it is not yet feasible to determine if the observed antivascular activity is of thermal origin only. Other bioeffects, including cavitation, radiation pressure and other non-linear effects, may also have had a role in disrupting tumor vascularity and reducing its cell numbers (Barnett et al. 1997; Barnett et al. 2000), and also in the blanching of the overlying skin. Thermal effects, however, explain the post-insonation clinical and histological changes observed in the skin overlying the tumor. Ultrasound is also likely to have been reflected from the rear skin surface and contributed to the heating of the tumor. Whether the increase in temperature of the tumor is caused by reflected or transmitted ultrasound is not known and requires further investigation. Absorption of the ultrasound beam at a bone-soft tissue interface would also have had a heating effect and resulted in the observed localized muscle necrosis adjacent to the femur. Optimization of the beam size, frequency, duty cycle and intensity may further enhance the observed effects on the tumor, whilst minimizing damage to the surrounding normal tissues.

In considering the development of cancer therapies, Folkman (2001) proposed that a tumor should be considered to have two cellular compartments—one containing the tumor cells and the other, the endothelial cells of the vascular structures within the tumor. For anti-cancer therapy to be efficacious each compartment may be selectively targeted.

It was observed in the above study that low intensity ultrasound particularly targeted the vascular structures within the tumor, whilst the variable effects on the tumor cell compartment appeared to be secondary to the resultant ischemia. Thus, combining insonation with other forms of therapy that specifically target the neoplastic cell compartment of the tumor is also contemplated. Such a combination of therapies may result in a more comprehensive and effective cancer therapy than if either treatment was used alone.

While the invention has been described in detail and with reference to specific examples thereof, it will be apparent to one skilled in the art that various changes and modifications can be made therein without departing from the spirit and scope thereof.

### REFERENCES

Wood et al. The antivascular action of physiotherapy ultrasound on murine tumors. Ultrasound in Medicine and Biology, 2005, 10, 1403-1410.
Bunte et al. Histpathological observations of the antivascular effects of physiotherapy ultrasound on a murine neoplasm. Ultrasound in Med. & Biol., 2006, 32 (3):453-461.
Wood et al. The antivascular actions of mild intensity ultrasound on a murine neoplasm. Therapeutic Ultrasound: 5th International Symposium on Therapeutic Ultrasound. Edited by G. T. Clement, N. J. McDannald, and K. Hynynen, American Institute of Physics, 2006:467-471.
Baker et al. A review of therapeutic ultrasound: biophysical effects. Phys Ther 2001; 81:1351-1358.
Barnett et al. The sensitivity of biological tissue to ultrasound. Ultrasound Med Biol 1997; 23:805-812.
Barnett S B, Ter Haar G R, Ziskin M C, et al. International recommendations and guidelines for the safe use of diagnostic ultrasound in medicine. Ultrasound Med Biol 2000; 26:355-366.
Carmeliet P, Jain R K. Angiogenesis in cancer and other diseases. Nature 2000; 407:249-257.
Carmeliet P, Conway E M. Growing better blood vessels. Nat Biotechnol 2001; 19:1019-1020.
Chomas et al. Contrast-enhanced US of microcirculation of superficially implanted tumors in rats. Radiology 2003; 229:439-446.
Clement G T. Perspectives in clinical uses of high-intensity focused ultrasound. Ultrasonics 2004; 42:1087-1093.
Demmink et al. The variation of heating depth with therapeutic ultrasound frequency in physiotherapy. Ultrasound Med Biol 2003; 29:113-118.
Diederich C J, Stafford R J, Nau W H, et al. Transurethral ultrasound applicators with directional heating patterns for prostate thermal therapy: in vivo evaluation using magnetic resonance thermometry. Med Phys 2004; 31:405-413.
Eikesdal H P, Bjerkvig R, Raleigh J A, Mella O, Dahl O. Tumor vasculature is targeted by the combination of combretastatin A-4 and hyperthermia. Radiother Oncol 2001; 61:313-20.
Fleischer A C. Sonographic depiction of tumor vascularity and flow: from in vivo models to clinical applications. J Ultrasound Med 2000; 19:55-61.
Folkman J. Tumor angiogenesis: therapeutic implications. N Engl J Med 1971; 285:1182-1186.
Folkman J. Angiogenesis. In: Braunwald E, Fauci A S, Kasper D L, Hauser S L, Longo D L, Jameson J L, eds. Harrison's textbook of internal medicine. New York: Mc Graw-Hill, 2001. p. 517-530.
Forsberg F. Ro R J, Potoczek M, et al. Assessment of angiogenesis: implications for ultrasound imaging. Ultrasonics 2004; 42:325-330.
Gee M S, Procopio W N, Makonnen S, et al. Tumor vessel development and maturation impose limits on the effectiveness of anti-vascular therapy. Am J Pathol 2003; 162:183-193.
Haroon Z A, Peters K G, Greenberg C S, Dewhirst M W. Angiogenesis and oxygen transport in solid tumors In: Teicher B A, ed. Antiangiogenic agents in cancer therapy. Totowa, N.J.: Humana Press, 1999. p. 3-21.
Hazle J D, Diederich C J, Kangasniemi M, et al. MRI-guided thermal therapy of transplanted tumors in the canine prostate using a directional transurethral ultrasound applicator. J Magn Reson Imaging. 2002; 15:409-417.
Hynynen et al. MR imaging-guided focused ultrasound surgery of fibroadenomas in the breast: a feasibility study. Radiology. 2001; 219:176-185.
Jain R K. Tumor angiogenesis and accessibility: role of vascular endothelial growth factor. Semin Oncol 2002; 29, Suppl 16:3-9.
Jin et al. Combination effect of photodynamic and sonodynamic therapy on experimental skin squamous cell carcinoma in C3H/HeN mice. J Dermatol 2000; 27:294-306.
Kamotani et al. Multigated contrast-enhanced power Doppler to measure blood flow in mice tumors. Ultrasound Med Biol 2003; 29:977-984.
Krix et al. Comparison of intermittent-bolus contrast imaging with conventional power Doppler sonography: quantification of tumor perfusion in small animals. Ultrasound Med Biol 2003; 29:1093-1103.
Madersbacher et al. tissue ablation by transrectal high intensity focused ultrasound: histological impact and clinical application. Ultrason Sonochem 1997; 4:175-179.
Melodelima et al. Trans-esophageal ultrasound applicator for sector-based thermal ablation: first in vivo experiments Ultrasound Med Biol 2003; 29:285-291.
Nishimura et al. Microangiographic and histologic analysis of the effects of hyperthermia on murine tumor vasculature. Int J Radiat Oncol Biol Phys 1988; 15:411-420.
Paterson et al. Laparoscopic partial kidney ablation with high intensity focused ultrasound. J Urol 2003; 169:347-351.
Palussiere et al. Feasibility of MR-guided focused ultrasound with real-time temperature mapping and continuous sonication for ablation of VX2 carcinoma in rabbit thigh. Magn Reson Med 2003; 49:89-98.
Robertson V J, Baker K G. A review of therapeutic ultrasound: effectivness studies. Phys Ther 2001:81:1339-1350.
Rowland et al. MRI study of hepatic tumors following high intensity focused ultrasound surgery. B J Radiol 1997; 70:144-153.
Sehgal et al. Quantitative vascularity of breast masses by Doppler imaging: regional variations and diagnostic implications. J Ultrasound Med 2000; 19:427-440.
Sehgal et al. Renal blood flow changes induced with endothelin-1 and fenoldopam mesylate at quantitative Doppler US: initial results in a canine study. Radiology 2001; 219:419-426.
Shattuck D P, von Ramm O T. Compound scanning with a phased array. Ultrason Imaging 1982; 4:93-107.
Uchida et al. Transrectal high-intensity focused ultrasound for treatment of patients with stage T1b-2N0M0 localized prostate cancer: a preliminary report. Urology 2002; 59:394-398; discussion 398-399.
Rosenthal I, et al. Sonodynamic therapy—a review of the synergistic effects of drugs and ultrasound. Ultrason Sonochem 2004; 11:349-363.
Tomizawa M, et al. Irradiation with ultrasound of low output intensity increased chemosensitivity of subcutaneous solid tumors to an anti-cancer agent. Cancer Lett 2001; 173:31-35.
Yu T, et al. A review of research into the uses of low level ultrasound in cancer therapy. Ultrason Sonochem 2004; 11:95-103.

What is claimed is:

1. A method for detecting and disrupting fragile blood vessels formed as a result of tumor angiogenesis by manipulating an extent of tissue vascularity in vivo, the method comprising:
targeting a region in need of manipulating the extent of vascularity of the tissue by a diagnostic imaging;
visualizing said fragile blood vessels in the tissue;
obtaining a characteristic of the tissue; and
contacting the tissue with a therapeutic ultrasound consisting of an unfocused or mildly focused ultrasound beam having an intensity below 2.6 W/cm², wherein the therapeutic ultrasound is emitted from an ultrasound unit in accordance with the characteristic of the tissue and thereby reducing the extent of vascularity of the tissue and disrupting said fragile blood vessels formed to support the tumor in the region of interest.

2. The method of claim 1, wherein the characteristic of the tumor is at least one of the size of the tumor, depth of the tumor from the skin and the vascularity of the tumor.

3. The method of claim 1, wherein the characteristic of the ultrasound treatment is at least one of the size of the ultrasound beam, frequency of sonication, duty cycle and a treatment time.

4. The method of claim 1, wherein said visualizing is performed by an imaging ultrasound.

5. The method of claim 4, wherein the imaging ultrasound has intensity of 0.5 W/cm² or less.

6. The method of claim 4, wherein said visualizing and said contacting the tissue with a therapeutic ultrasound are conducted simultaneously, provided that the frequency of the imaging ultrasound and the frequency of the therapeutic ultrasound do not overlap.

7. The method of claim 4, wherein chemotherapeutic compounds are not used.

8. The method of claim 1, wherein tissue vascularity is reduced by at least 10%.

9. The method of claim 1, wherein the change in tissue vascularity is visualized and said contacting the tissue with a therapeutic ultrasound is repeated until a desired degree of tissue vascularity is achieved.

10. A device for detecting and manipulating tissue vascularity, the device comprising:
an imaging unit for imaging of an area of a tumor;
a processing unit for determining a characteristic of the tumor;
a processing unit for determining a characteristic of an ultrasound treatment correlated to the characteristic of the tumor; and
an ultrasound treatment unit capable of generating an unfocussed or mildly focused ultrasound beam having an intensity below 2.6 W/cm² which is sufficient to disrupt blood vessels formed to support the tumor but not blood vessels formed to support healthy tissues.

11. The device of claim 10, wherein the imaging unit comprises an imaging ultrasound.
